(19) United States
(12) Patent Application Publication
(10) Pub. No.: US 2010/0210898 A1
(43) Pub. Date: Aug. 19, 2010

(54) MAMMALIAN REPRODUCTION

(75) Inventors: Ray Noble, Ayr (GB); Tom Gilcrist, Ayr (GB); Alex Willis, Ayr (GB); Ian Miller, Kilmarnock (GB)

Correspondence Address: SPECKMAN LAW GROUP PLLC, 1201 THIRD AVENUE, SUITE 330, SEATTLE, WA, 98101, US

(73) Assignee: DALRIADA PRODUCTS LIMITED, Ayr (GB)

(21) Appl. No.: 12/664,306

(22) PCT Filed: Jun. 9, 2008
(86) PCT No.: PCT/GB2008/001957

§ 371 Date: Apr. 27, 2010

(30) Foreign Application Priority Data
Jun. 11, 2007 (GB) 0711312.9

(51) Int. Cl. A61B 17/425

(52) U.S. Cl. 600/33

(57) ABSTRACT

The invention is designed to enhance mammalian reproduction and in particular to improve the use of existing artificial insemination catheters in combination with novel products and processes for the insemination of mammals such as pigs. The invention comprises a controlled/sustained release delivery system comprising a metabolite/carrier combination based on specific gels, polymers and/or other molecular complexes for metabolite delivery at the point of insemination in order to promote aspects of spermatozoa function.

### FIELD OF THE INVENTION

The present invention relates to improvements in and relating to mammalian reproduction and in particular to improvements which utilize artificial insemination catheters in combination with novel products and processes for the insemination of mammals such as pigs.

### BACKGROUND

In mammalian species the process of reproduction can be simply described as fertilisation occurring through binding and fusion of the male gamete (spermatozoa) with the female gamete (oocyte) following the former's deposition within the female genital tract. In nature this occurs through natural mating. However within the intensive animal production systems that prevail in advanced societies, natural mating has largely been superseded by assisted reproduction techniques (artificial insemination, AI). This is seen as being of paramount importance to maintain both genetic improvement and economic production/competitiveness.

With the contemporary development of large-scale livestock programmes based on AI, the need for the transport of semen from point of collection to the site of insemination and the necessity to cover large numbers of females over undecided periods at differing times of the year require the preservation of spermatozoa under artificial conditions for periods ranging from days to weeks. As a result, problems have to be faced with respect to the maintenance of sperm function and fertilisation capacity at point of insemination. Of particular concern following semen storage is the significant decline in spermatozoa motility, their forward progression and a reduction in capacitation, that is, the subsequent ability of the spermatozoa to fuse with the ovum.

These are features of paramount importance for achieving adequate levels of reproduction. Of particular contemporary interest are the fundamental mechanisms that have an ability to “switch on” these prime controlling features of reproductive performance. A range of major metabolites involved in reproduction have been identified but their effective application under practical conditions has yet to be overcome. If able to be put into practice the combined consequences on motility and subsequent fertilisation could be immense, not only in terms of “in field” situations but also under particular insemination circumstances where spermatozoa availability and insemination success are at a premium e.g. paucity of particular genetic material, sexual pre-selection of spermatozoa.

The introduction of any metabolite into the insemination process always poses particular problems. Energy levels and associated mechanisms within the spermatozoa are of a finite nature and although storage media are designed to reduce spermatozoa vigour and conserve metabolic features, there is an inevitable significant erosion of available energy and associated metabolic processes. Thus to introduce any metabolite for the promotion of motility at a time other than at point of insemination would be counter to any degree of success. Similarly the introduction of any metabolite to promote capacitation would bring about a premature capacitation. Further impositions also have to be taken into consideration. Thus any metabolite must be introduced at a uniform rate into the seminal infusate to give an equal exposure to all spermatozoa. The metabolite must be able to exert an influence throughout the female reproductive tract and be compatible with both the physical and biochemical characteristics of the spermatozoa. In practical terms the methodology must be maximally “user friendly” to the operator and recipient animal alike, whilst cognizance of specific specie-based problems have also to be given e.g. in the pig stimulation of both motility and capacitation would have to cover an extended multi-ova release.

### SUMMARY OF THE INVENTION

In accordance with the present invention there is provided a controlled/sustained release delivery system comprising a metabolite/carrier combination based on specific gels, polymers and/or other molecular complexes for metabolite delivery at the point of insemination in order to promote aspects of spermatozoa function.

The present invention is of particular use where semen has been stored for an extended period.

A range of metabolites have been identified that have the ability to stimulate motility, its characteristics and capacitation.

The invention describes, in one aspect, an innovative approach for the delivery of such stimulatory metabolites, individually or in combination.

Preferably, the invention comprises a semi-solid insert placed within the distal end of an AI catheter.

Preferably, the semi-solid insert comprises a gel composed of a combination of methyl ester pectins.

The invention involves an incorporation/binding of metabolites of known promotional activity to stimulate major parameters of spermatozoa function, in particular motility, their motile characteristics, and capacitation.

The invention described involved the delivery of a combination of caffeine and CaCl₂ (Ca²⁺) to promote spermatozoa motility and motile characteristics and capacitation respectively.

The physical characteristics of the insert were such as to provide immobility within the inseminating device during storage, but its immediate mobilisation and transfer as a complete entity plus metabolites into the female genital tract (uterus) in the inseminating media at point of insemination.

The chemical properties of the insert were such as to enable a slow but regulated dissolution within the female tract (uterus) to facilitate a modulated slow release of the metabolites over a chosen extended period of time.

Modulated release can be a substantially constant rate of release over a predetermined time. In addition, the rate of release can be varied in any given carrier by the selection of more than one gel (each of which have different rates of dissolution) to form the carrier.

Metabolite release from the insert and the subsequent effects upon spermatozoa characteristics were tested in vitro with pig spermatozoa from pedigree breeding boars and its efficacy on fertility by in field inseminations.

Metabolite release showed significant positive effects upon spermatozoa motility and associated characteristics compared to controls.

Positive effects of the caffeine release on the spermatozoa characteristics remained evident for a full 24 hour period of the incubation.

The practical efficacy of the modified insemination catheter was determined by in field trials (still ongoing) on pedigree breeding sows.

In production terms use of the modified insemination catheter resulted in extra piglets per 100 inseminations in the range 44-387 compared to the use of standard catheters as a result of a combination of an increased farrowing rate and total births.

The invention is applicable for the modulated delivery of combinations/permutations of a range of metabolites identified with an ability to promote spermatozoa characteristics at point of insemination for the enhancement of fertilisation.

The invention is applicable to a range of animals other than the pig where AI is of major consideration and economic importance.

The invention is applicable to improving the reproductive efficiency for a range of specialist AI methodologies e.g. deep in utero insemination, reduced spermatozoa number, spermatozoa following X/Y sexing.

The present invention provides a controlled/sustained release delivery device for mammalian artificial insemination as further defined in the attached claims.

### BRIEF DESCRIPTION OF THE DRAWING

FIG. 1 is a side view of an AI catheter containing a device in accordance with the present invention.

In accordance with the present invention, a range of experimental work was conducted. The primary objectives of the work undertaken were as follows.
(i) The investigation, both theoretically and practically, of a suitable system for the modulated delivery of (a) a metabolite for the purpose of stimulating the motility and forward progression of spermatozoa following an extended period of semen storage and (b) a metabolite for the induction of capacitation.
(ii) The evaluation of options for compatibility with standard insemination devices and in field application to metabolite delivery at point of insemination.
(iii) The evaluation/quantification of the chosen system in terms of compatibility with semen AI infusate and the modulated release/delivery of the metabolites into the seminal fluid infusate.
(iv) The modulated release/delivery of the metabolites into the in utero fluid environment.
(v) The quantification of metabolite release over sequential time periods.
(vi) The evaluation of such modulated metabolite release on major spermatozoa features.
(vii) In field testing of the delivery system and evaluation in terms of fertility and reproductive performance.
(viii) Technical and commercial evaluation of the system in terms of market acceptability.

### EXPERIMENTAL PROCEDURES, DESCRIPTIONS AND OUTCOMES

### Procedures

Investigations were performed on pig semen obtained from large white/landrace stud boars housed at established pedigree and commercial breeding complexes. The choice of the pig for experimentation was based on the fact that amongst intensive animal stock, spermatozoa from the boar presents by far the most problematic with respect to negative effects arising from storage in terms of deterioration of quality, motility and subsequent fertilising capability to cover the extended multi-ovulatory female reproductive system.

Qualitative and quantitative analytical data involved the extensive use of standard and state of the art laboratory methodologies e.g. spectrophotometry, capillary gas liquid chromatography, high performance liquid chromatography, cell number and integrity evaluations by differential staining and motility parameters by computer-aided spermatozoa analysis (CASA)

Reported in-field data on parameters of fertility (insemination success, live and dead births etc) were obtained on the outcomes of the insemination, standard control and experimental, performed at the breeding complexes with inseminations spread over a period of two to three months (July-September) in all cases, and subsequent progeny born October-December. Sows allocated either to a treated or control group were balanced for parity number (number of litters) and breed. All sows were inseminated twice (am zero and 24 hours) according to established practice using a 75 ml dose of semen, this being collected from the same boar and distributed evenly across the two groups. Comparative data collected from the in-field investigations were:
(a) Farrowing rate percentage i.e. The number of sows that actually farrowed a litter of piglets following AI.
(b) Total piglets born i.e. The number of piglets both alive and dead at farrowing.
(c) Interpolation of such data in terms of commercial benefit from treatment.

### DESCRIPTION AND RESULTS

### The System (General)

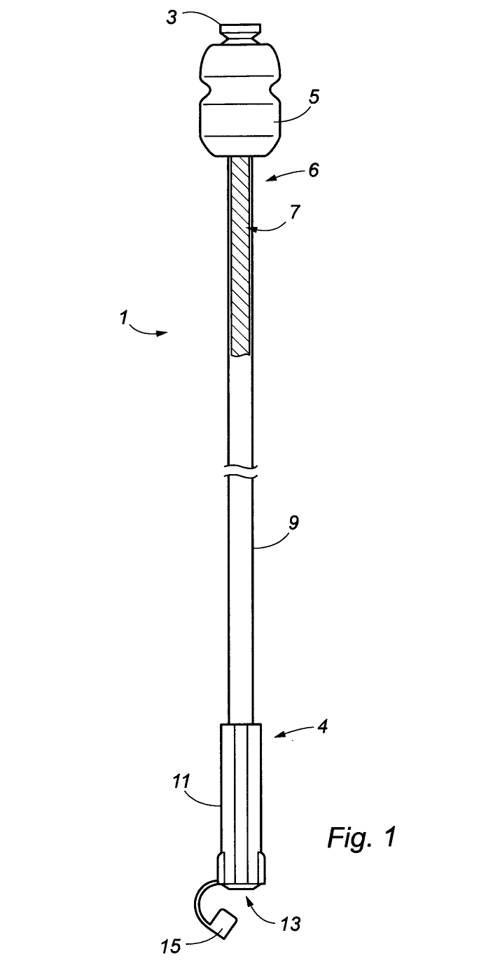

FIG. 1 shows a standard universal pig insemination catheter 1 which comprises an elongated cylindrical 0.5 m tube 9 with a proximal end 4 and a distal end 6. The proximal end 4 supports a plastic cover 11 which has a stopper 15 adapted for insertion into the end of the tube 9. The distal end 6 of the catheter 1 comprises a bulbous head 5 having a stopper 3 adapted for insertion into the end of the tube. The device of the present invention 7 comprising the carrier and metabolite (also termed the controlled release insert/plug) is placed in the tube 9 at or near the distal end thereof and occupies the space within the bulbous head of the insemination catheter around 2-3 cm from the end of the plastic tube. The controlled release insert can be poured into the tube 9 as a predetermined quantity of liquid which cools to form a gel or the controlled release insert can be preformed and inserted into the tube 9.

In use, around 75 ml of inseminate (spermatozoa plus diluent) is delivered through the tube 9. The diluent can embrace a full range of standard commercial products. In addition, AI catheter can be of a standard type and any modification of a standard catheter for the purposes of the present invention that would not interfere with or complicate established insemination procedures.

In one preferred example of the present invention, a controlled release insert consisting of a 6 percent (w/w) aqueous gel of high methyl ester pectins was used. The chosen active metabolites within the insert were caffeine to promote spermatozoa motility and forward progression and Ca²⁺ (provided as CaCl₂) to promote capacitation.

The controlled release insert in accordance with the invention is designed to provide a necessary combination of physical immobility commensurate to storage at 15-20 C prior to use and immediate mobility as a single entity when exposed to an inseminating fluid for transfer into the female genital tract (uterus). In general, the controlled release insert has been designed such that its viscosity allows it to remain positioned at a predetermined place (usually the distal end of an AI tube) when not in use, but also to easily move into the uterus of a mammal once the seminal fluid is introduced into the tube.

In addition, it is preferred that the controlled release insert display some degree of elasticity that it can retain its shape when placed under stress.

The controlled release insert may also comprise a thermosetting gel. This will allow a user to inject the gel into an AI tube as a liquid which will set into a gel once the liquid cools.

The controlled release insert is designed to provide an immediate slow release/delivery of the metabolites once within the genital tract (37 C) under the combined influences of the continued presence of insemination fluid and natural tissue secretions over an extended time period sufficient to cover ovulation. The slow release is provided when the gel dissolves in the uterus, this type of release is in preference to gels where liquid leaches out. As leaching effectively dries out the gel, it can result in a hard low water content gel being left as a residue in the uterus. In addition, a proportion of the metabolite will remain in the gel in these circumstances.

In cases where pectin gels have been used, it has been found that the presence of the metabolite alters the properties of the pectin gel such that, in the absence of metabolites, the gel can be re-melted (thermoreversible). However, when metabolites are present, the gel is not thermoreversible.

Table 1 shows the results obtained on the ability of the device to release caffeine and Ca²⁺ over sequential 30 minute periods of exposure to a standard BTS diluent at 37 C. The effect of caffeine release on the motile characteristics of spermatozoa were measured sequentially during a 24 hour period of incubation at 37 C of the insert in 75 ml of BTS diluent containing 2 billion spermatozoa i.e. Equivalent to a standard single insemination. A significant effect on motility was observed throughout the full 24 hours. Thus comparative results for spermatozoa motility at 24 hours following caffeine release were: prior to caffeine exposure i.e. At time zero, 70-75%; controls (no caffeine exposure) 60-650; following caffeine exposure, 85-90%.

Table 2 shows production performance data obtained from a comparative in-field investigation involving a general stock breeder and a pedigree stock breeder. Insemination with the catheter modified to contain the device in accordance with the present invention improved the farrowing rate and piglet output in both circumstances, giving rise to 112 extra piglets per 100 sows in the case of the general stock breeder and, in spite of very high levels of production performance, 44 extra piglets per 100 sows in the case of the pedigree stock breeder.

TABLE 1
Rates of release (% of initial total incorporated)
of caffeine and calcium from the Pectin insert following
incubation in BTS diluent at 37° C.

| | Time (hrs) | | | | | |
|---|---|---|---|---|---|---|
| | 0.25 | 0.5 | 0.75 | 1.00 | 1.25 | 1.50 |
| % Caffeine and Calcium | 5 | 25 | 53 | 65 | 74 | 81 |
| Release | | | | | | |
| | Time (hrs) | | | | | |
| | 1.75 | 2.00 | 2.30 | 3.00 | 4.00 | 6.00 |
| % Caffeine and Calcium | 84 | 85 | 87 | 90 | 94 | 100 |
| Release | | | | | | |

TABLE 2
Production performance data for a device in
accordance with the present invention used in an AI catheter
showing production/performance benefits for general and
pedigree stock breeding in using the device according to the
present invention.

| | |
|---|---|
| General Stock | Pedigree |
| Breeder | Stock Breeder |

| | Control | Treated | Control | Treated |
|---|---|---|---|---|
| Farrowing Rate % | 82 | 88 | 89 | 91 |
| Piglets/Sow | 12.8 | 13.2 | 12.8 | 13 |
| Total No. of | 1050 | 1162 | 1139 | 1183 |
| piglets/hundred | | | | |
| sows | | | | |

| | | |
|---|---|---|
| Extra | 112 | 44 |
| piglets/100 | | |
| sows | | |

| | |
|---|---|
| Mean | 78 |
| benefit/hundred | |
| sows | |
| Mean | 0.78 |
| benefit/sow | |

Tables 3 and 4 below shows additional trials at 4 general stock breeding farms where similar but improved benefits to those of table 2 were recorded.

| | |
|---|---|
| Farm1 | Farm 2 |

| | Control | Treated | Control | Treated |
|---|---|---|---|---|
| Farrowing Rate % | 80 | 90 | 85 | 96 |
| Piglets/sow | 11 | 12 | 9 | 12 |
| Total number of | 880 | 1080 | 765 | 1152 |
| piglets/100 sows | | | | |

| | | |
|---|---|---|
| Extra | 200 | 387 |
| piglets/100 sows | | |

| | |
|---|---|
| Farm 3 | Farm 4 |

| | Control | Treated | Control | Treated |
|---|---|---|---|---|
| Farrowing Rate % | 80 | 83 | 83 | 89 |
| Piglets/sow | 11 | 12 | 11 | 12 |
| Total number of | 880 | 996 | 913 | 1068 |
| piglets/100 sows | | | | |

| | | |
|---|---|---|
| Extra | 116 | 155 |
| piglets/100 sows | | |

The mean benefit per 100 sows for farms 1-4 is 233 extra piglets

The mean benefit per sow for farms 1-4 is 2.33 extra piglets

The carrier should provide immobility within the inseminating device during storage, but its immediate mobilisation and transfer as a complete entity plus metabolites into the female genital tract (uterus) in the inseminating media at point of insemination. In addition, the carrier should enable a slow but regulated dissolution within the female tract (uterus) to facilitate a modulated slow release of the metabolites over a chosen extended period of time.

A number of polymers including polysaccharides could be used to provide a carrier in a device in accordance with the present invention. A wide selection of permutations/combinations of polysaccharides were tried all of which displayed varying abilities to satisfy these requirements.

The following gels and gel formers are examples of the types of carriers that could be used:

Gellan gums;

### Alginates;

gelatin/maize starch; and
xanthan/locust bean gum.

In general, a preferred gel should demonstrate at least some of the following properties to some degree:

elasticity; plasticity; thermosetting; soluble, biodegradable; and biocompatible.

In another embodiment of the invention, the carrier comprises two gels having different compositions and which release the metabolites at different rates.

In addition, the choice of a simple admixture of caffeine and Ca²⁺ in the above example of the present invention was based on well substantiated data for their respective promotional abilities for spermatozoa motile characteristics and capacitation.

The carrier, however, is well capable of incorporating and releasing in a controlled manner a range of both organic and inorganic metabolites with similar promotional abilities (e.g. steroids, acetylated glyco-phospholipids, albumin, xanthine as derivatives, peptide-based hormones and various semi micro/micro cations.

The present invention has the ability to gear the release of any promotional metabolite to suit a range of specific situations, for example in terms of
1. complementation to standard insemination devices.
2. time and space requirements e.g. coincident to time of insemination, placement within the female genital tract, single/multi ovulatory system.
3. appropriate metabolite release e.g. absolute level, pulsed or continuous.
4. homogeneity of introduction.
5. idealised mode of activation for metabolite release e.g. physical, chemical triggering.

The successful outcome to such a delivery system would not only be uniquely innovative and therefore highly commecialisable but also has the potential for spin-offs to other important areas of the fertility sector such as human AI and the survival of fertilised ova following in vitro fertilisation.

Improvements and modifications may be incorporated herein without deviating from the scope of the invention.

1. An inseminating device comprising a controlled/sustained release delivery insert for mammalian artificial insemination, the insert comprising a carrier incorporating a metabolite, the carrier comprising a polymer adapted to deliver the metabolite at the point of insemination in order to promote aspects of spermatozoa function within semen, said insert adapted to be maintained with the inseminating device during storage but allowing mobilization and transfer into the female genital tract (uterus) in the inseminating media at the point of insemination.

2. An inseminating device as claimed in claim 1 wherein the carrier immobilizes the metabolite whilst stored, but mobilises the metabolites upon entry into the female genital tract (uterus).

3. An inseminating device as claimed in claim 1 wherein the carrier dissolves within the female tract (uterus) to facilitate release of the metabolite over a chosen period of time.

4. An inseminating device as claimed in claim 3 wherein the release is modulated.

5. An inseminating device as claimed in claim 1 wherein the carrier comprises two or more types of polymer which are incorporated into the carrier and which dissolve within the female tract at differing rates.

6. (canceled)

7. An inseminating device as claimed in claim 1 wherein the device is adapted for insertion into a distal end of an artificial insemination catheter.

8. An inseminating device as claimed in claim 1 wherein the carrier comprise a gel.

9. An inseminating device as claimed in claim 8 wherein the gel is a thermosetting gel.

10. An inseminating device as claimed in claim 8 wherein the gel is adapted to dissolve at or near normal mammalian body temperature.

11. An inseminating device as claimed in claim 8 wherein the gel is adapted to dissolve when in contact with an inseminating media.

12. An inseminating device as claimed in claim 8 wherein the gel comprises a polysaccharide.

13. An inseminating device as claimed in claim 12 wherein the gel comprises a combination of methyl ester pectins.

14. An inseminating device as claimed in claim 1 wherein the carrier comprises a gel former.

15. An inseminating device as claimed in claim 1 wherein the metabolite stimulates motility.

16. An inseminating device as claimed in claim 1 wherein the metabolite stimulates capacitation.

17. An inseminating device as claimed in claim 1 wherein the metabolite is selected from the group consisting of: caffeine; Ca⁺; steroids; acetylated glyco-phospholipids; albumin; xanthine derivatives; peptide-based hormones; and semi micro/micro cations.

18. An inseminating device as claimed in claim 17 wherein the metabolite is Ca²⁺ ions.

19. A method for the artificial insemination of mammals comprising the steps of:
preparing an inseminating device as claimed in claim 1; and
inseminating the mammal with the loaded catheter inseminating device.

20. An inseminating device as claimed in claim 1 in the form of a catheter.

21. An inseminating device as claimed in claim 1, wherein the insert and the inseminating media are transferred into the female genital tract substantially homogenously.
